SEQUENCE LISTING (17 sequences)

SEQ ID NO: 1 - DNA, 736 bp, Homo sapiens
  source 1..736: mol_type = genomic DNA
agagcagaggaaagacctgacgcgcctcagtaagcatgtggaggccctcagggcccagctggaggaggctgaagggcagaaggatggcctgaggaagcaggcgggcaagctggagcaggcgctgaaacaggagcagggggcacggcggcgacaggcggaggaggatgagcagtgcctgtctgagtgggagcacgacaaacagcagctgctcacagaaacaagtgacctaaagacaaagatggccaccctggagagagaactgaaacagcagcgggagtccacacaggctgtggaggcaaaggcccagcagctgcaggaggaaggtgagcgcagggcggcagcggagaggcaggtgcagcagctggaggagcaggtgcagcagttggaggcgcaggtgcagctgttggtgggtcggctggagggcgctggccagcaggtctgctgggccagcacggagctggataaggagaaggcccgtgtcgacagcatggtccgccaccaggagtctctgcaggccaagcagcgagccctgctaaagcagctggacagcctggaccaggaacgtgaggagctgcggggcagcctggacgaggctgaggcccagcgggcccgcgtggaggagcagctgcagagcgagcgggagcaggggcaatgccagctcagggcccagcaggagctgctgcagagcctgcagagggagaagcaaggcctggagcaggcgactacggacctgcgg

SEQ ID NO: 2 - protein, 276 aa, Homo sapiens
AEQRKDLTRLSKHVEALRAQLEEAEGQKDGLRKQAGKLEQALKQEQGARRRQAEEDEQCLSEWEHDKQQLLTETSDLKTKMATLERELKQQRESTQAVEAKAQQLQEEGERRAAAERQVQQLEEQVQQLEAQVQLLVGRLEGAGQQVCWASTELDKEKARVDSMVRHQESLQAKQRALLKQLDSLDQEREELRGSLDEAEAQRARVEEQLQSEREQGQCQLRAQQELLQSLQREKQGLEQATTDLRLTILELERELEELKERERLLVAFPDLHRPT

SEQ ID NO: 3 - DNA, 20 bp, synthetic construct
  misc_feature 1..20: note = primer were designed for the two conserved functional domains ofCCDC157, the SMC domain and the APC superfamily domain.
  source 1..20: mol_type = other DNA
gaacctgccctcgtccttag

SEQ ID NO: 4 - DNA, 20 bp, synthetic construct
  misc_feature 1..20: note = primer were designed for the two conserved functional domains ofCCDC157, the SMC domain and the APC superfamily domain.
  source 1..20: mol_type = other DNA
atcacagtgtcaccaccgga

SEQ ID NO: 5 - DNA, 20 bp, synthetic construct
  misc_feature 1..20: note = primer were designed for the two conserved functional domains ofCCDC157, the SMC domain and the APC superfamily domain.
  source 1..20: mol_type = other DNA
aggtgtagacacatggcagc

SEQ ID NO: 6 - DNA, 20 bp, synthetic construct
  misc_feature 1..20: note = primer were designed for the two conserved functional domains ofCCDC157, the SMC domain and the APC superfamily domain.
  source 1..20: mol_type = other DNA
aaaggtgagtcaacccccac

SEQ ID NO: 7 - DNA, 20 bp, synthetic construct
  misc_feature 1..20: note = primer were designed for the two conserved functional domains ofCCDC157, the SMC domain and the APC superfamily domain.
  source 1..20: mol_type = other DNA
tacccttccacgcatgtgac

SEQ ID NO: 8 - DNA, 20 bp, synthetic construct
  misc_feature 1..20: note = primer were designed for the two conserved functional domains ofCCDC157, the SMC domain and the APC superfamily domain.
  source 1..20: mol_type = other DNA
ggctttaacacccttgctgc

SEQ ID NO: 9 - DNA, 21 bp, Homo sapiens
  source 1..21: mol_type = genomic DNA
gcaggtgcagcagctggagga

SEQ ID NO: 10 - protein, 37 aa, Homo sapiens
RAAAERQVQQLEEQVQQLEAQVQLLVGRLEGAGQQVC

SEQ ID NO: 11 - protein, 36 aa, Homo sapiens
GGSGEAGAAVGGAGAAVGGSAGGRWPAGLLGQHGAG

SEQ ID NO: 12 - DNA, 23 bp, synthetic construct
  misc_feature 1..23: note = Primers to identify mouse mutant genotypes.
  source 1..23: mol_type = other DNA
ctctgagagcggcctatggtggg

SEQ ID NO: 13 - DNA, 23 bp, synthetic construct
  misc_feature 1..23: note = Primers to identify mouse mutant genotypes.
  source 1..23: mol_type = other DNA
gggaggatccatccaacctaggg

SEQ ID NO: 14 - DNA, 22 bp, synthetic construct
  misc_feature 1..22: note = Primers to identify mouse mutant genotypes.
  source 1..22: mol_type = other DNA
gctctgcctccttctgagttag

SEQ ID NO: 15 - DNA, 22 bp, synthetic construct
  misc_feature 1..22: note = Primers to identify mouse mutant genotypes.
  source 1..22: mol_type = other DNA
gtttgtcttctgaccacactcc

SEQ ID NO: 16 - DNA, 22 bp, synthetic construct
  misc_feature 1..22: note = Primers to identify mouse mutant genotypes.
  source 1..22: mol_type = other DNA
ctcgtctcaataaacatgtggg

SEQ ID NO: 17 - DNA, 22 bp, synthetic construct
  misc_feature 1..22: note = Primers to identify mouse mutant genotypes.
  source 1..22: mol_type = other DNA
ccacctttgtctttaggtcaca

(19) United States
(12) Patent Application Publication
(10) Pub. No.: US 2023/0050804 A1
(43) Pub. Date: Feb. 16, 2023

(54) USE OF CCDC157 GENE AND MUTANT GENES THEREOF AS MOLECULAR MARKERS IN DIAGNOSIS OF MALE INFERTILITY DISEASES

(71) Applicant: ZHEJIANG UNIVERSITY, Hangzhou (CN)

(72) Inventors: Yongmei XI, Hangzhou (CN); Huimei ZHENG, Hangzhou (CN); Chen LI, Hangzhou (CN); Xiaohang YANG, Hangzhou (CN); Jingping LI, Hangzhou (CN); Fengbin ZHANG, Hangzhou (CN); Zhongyan LIANG, Hangzhou (CN); Jinggen WU, Hangzhou (CN); Xin YUAN, Hangzhou (CN); Jiaru HOU, Hangzhou (CN); Xinhai ZHU, Hangzhou (CN); Huan DENG, Hangzhou (CN)

(21) Appl. No.: 17/866,582

(22) Filed: Jul. 18, 2022

Related U.S. Application Data
(63) Continuation of application No. PCT/CN2021/095087, filed on May 21, 2021.

(30) Foreign Application Priority Data
Jul. 7, 2020 (CN) 202010646475.3

(51) Int. Cl. G01N 33/68

(52) U.S. Cl. CPC G01N 33/689; G01N 33/6818; G01N 2800/367

(57) ABSTRACT

The present application provides use of a CCDC157 gene and mutant genes thereof as molecular markers in diagnosis of male infertility diseases. Experiments have shown that the CCDC157-MIF515 mutant gene/protein causes male infertility, spermatogenesis disorder, sperm dysfunction, reduced sperm count, reduced sperm motility, abnormal sperm morphology, abnormal sperm head, etc. The CCDC157-MIF515 mutant gene/protein of the present application can be used as target genes for diagnosing male infertility. Meanwhile, the expression level of the CCDC157 of CCDC157 gene/protein is significantly reduced in NOA patients and SCOS patients, and the male infertility can be prevented and/or treated by increasing the activity and/or expression of the CCDC157 protein.

### TECHNICAL FIELD

The present application belongs to the field of biomedicine, more specifically belongs to the field of diagnosis of gene mutations, and specifically relates to a kit for diagnosing male infertility caused by a nucleotide sequence or amino acid sequence of CCDC157 mutation.

### BACKGROUND

About 15% of couples in the world have infertility problems, of which 50% are male factors, with a trend of increasing year by year. Male infertility has brought a heavy burden to individuals, families and the society. The causes of male infertility are complex, including anatomical structure and functional factors of the reproductive system, infection factors, endocrine and immune factors, genetic factors, mental and psychological factors, etc. The testicular spermatogenesis dysfunction caused by genetic defects accounts for about 10% to 15% of male infertility, and the main clinical manifestation is oligoasthenospermia (OAT) or azoospermia. Some patients with OAT and azoospermia can obtain their offspring through assisted reproductive technology (ART), but for male infertility or abnormal sperm caused by genetic defects, the ART may cause vertical transmission to affect the reproductive health of the offspring.

At present, the common method for determining male fertility is still routine semen analysis (including sperm count, viability, motility, morphology, etc.) combined with serum hormone level analysis, but its ability to predict male fertility is very limited. The routine semen analysis results of nearly 30% of male infertility patients are normal/near normal in clinic. The routine semen analysis can only reflect the most basic sperm quality, but cannot reflect other characteristics and functions of sperm. Therefore, it is necessary to identify new male fertility biomarkers to assist traditional routine semen analysis.

CDC157 protein belongs to the coiled-coil domain containing (CCDC) protein family, and it is conserved among multiple species and highly expressed in mammalian testes. The CCDC157 has only been reported to be an important factor in the fusion of a transport carrier and a Golgi apparatus, but its biological function and related molecular mechanism are still unclear.

### SUMMARY

The objective of the present application is to provide a CCDC157-MIF515 mutant gene/protein produced by new mutation of a CCDC157 gene on a third exon, the nucleotide sequence of which shows GC deletions on positions 15488-15489 and base deletions on positions 15507-15527 of a CCDC157 gene sequence; or the amino acid sequence of which shows that R is mutated to Q in the amino acid on position 387 of a CCDC157 polypeptide, and the amino acids on positions 388-424 are mutated to an amino acid sequence shown in SEQ ID NO.11, and finally the amino acid W on position 425 is mutated to a terminator. Disclosed is use of the CCDC157-MIF515 mutant gene/protein as a biomarker in the preparation of a kit for diagnosing male infertility, which provides a new biomarker for the existing kit for diagnosing male infertility and a new target for treating male infertility.

The present application further provides a kit for diagnosing male infertility, which includes a reagent for detecting a nucleotide sequence shown in SEQ ID NO. 1 or an amino acid sequence shown in SEQ ID NO. 2.

As a preferred solution, the reagent includes 3 pairs of primers, and the nucleotide sequences of the primers are shown in SEQ ID NO. 3 to SEQ ID NO. 8.

As a preferred solution, the kit further includes a carrier recording a judgment criterion; the judgment criterion may be: if the CCDC157-MIF515 mutant gene/protein is present in the sperm of a test subject, the test subject is or is suspected to be a male infertile patient.

The present application further provides use of a CCDC157 gene/protein test reagent in the preparation of a kit for diagnosing male infertility.

Further, the test reagent is used for treating a CCDC157 protein or a CCDC157 DNA, mRNA, or RNA sequence or miRNA targeting CCDC157.

The present application further provides use of the CCDC157 gene/protein as a drug target in the preparation of a product, and the product may have at least one of the following functions A1) to A9):

A1) preventing male infertility; A2) treating male infertility; A3) treating sperm dysfunction; A4) promoting sperm maturation; A5) improving sperm motility; A6) increasing sperm count; A7) repairing abnormal sperm morphology; A8) repairing a sperm head structure; and A9) improving male fertility.

Experiments have proved that the CCDC157-MIF515 mutant gene/protein will decrease sperm count, reduce motility, and cause abnormal sperm morphology. The male infertility can be diagnosed by testing whether the CCDC157-MIF515 mutant gene/protein is produced. The present application has great application value. Meanwhile, the expression level of the CCDC157 of CCDC157 gene/protein is significantly reduced in NOA patients and SCOS patients, and the male infertility can be prevented and/or treated by increasing the activity and/or expression of the CCDC157 protein.

### BRIEF DESCRIPTION OF DRAWINGS

The present application will be further illustrated below in conjunction with the drawings and embodiments.

FIG. 1 shows a screened patient with CCDC157-MIF515 mutation. (A) The CCDC157 sequencing analysis on the patient shows that the upper arrow indicates an abnormal site in a DNA sequence, and the lower arrow indicates that the CCDC157-MIF515 mutation leads to an early termination codon site in an amino acid sequence. (B) Analysis on sperm concentration of the patient compared with a normal control (p=0.0009). (C) Total vitality of the patient compared with the normal control (p=0.047). (D) Forward motile sperm of the patient compared with the normal control (P=0.043). (E) Percentage of normal sperm in the patient compared with the normal control (P=0.0002). (F) Sperm photo of the patient's sperm.

FIG. 2 shows the construction of CCDC157 mutant mice. (A) Gene knockout strategy; wild-type C57BL/6N background strain mice are designed with two-way gRNA near exon 4 and exon 9, and the identification primer positions are marked in the figure. (B) PCR identification on CCDC157 gene of F1 generation mice, any band cannot be amplified with primers P1 in wild-type mice, and CCDC157 mutant allele can be amplified with 444 bp bands in this system. (C) Analysis on sequencing results, the knockout of the CCDC157 gene results in a 4985 bp deletion and an 11 bp insertion. (D) CCDC157 protein framework; the CCDC157 mutant allele terminates after the 84th leucine. (E) PCR identification on CCDC157 gene of F2 mice, any band cannot be amplified with primers P1 in wild-type mice, and 904 bp bands can be amplified with primers P2. (F) Appearance of F2 mice. (G) Western Blot identification on the expression of CCDC157 protein in the protein extract of mice testicular tissues. −/− represents CCDC157 knockout homozygous mice, and +/+ represents wild-type mice.

FIG. 3 shows the comparison of male reproductive systems of CCDC157 knockout homozygous mice and wild-type mice. (A) Gonads of adult male mice dissected. (B) Analysis on testis weight of mice (P=0.4804). (C) Analysis on epididymis weight of mice (P=0.0019). (D) Analysis on seminal vesicle weight of mice (P=0.0031). (E) Analysis on sperm count in the tail of epididymis of mice (P=0.0006). (F) Analysis on percentage of motile sperm in mice (P<0.0001). (G) Optical microscope image of mice. (H) Percentage of abnormal sperm (P=6.13784E-11). −/− represents CCDC157 knockout homozygous mice, and +/+ represents wild-type mice.

FIG. 4 shows the comparison of sperm in the tail of epididymis of CCDC157 gene knockout homozygous mice and wild-type mice. (A, B) HE stained epididymal tails of mice. A is a wild-type mouse, and B is a CCDC157 knockout mouse; arrows in A indicate the head morphology of typical normal sperm, and arrows in B indicate the head structure of abnormal sperm. (C) Electron micrograph of epididymal tail sperm under low power microscope; the quantity of sperm in the sperm epididymis of CCDC157 mutant mice is significantly less than that in wild-type mice. (D) Electron micrograph of the middle segment of mice sperm flagella. (E) Electron micrograph of the main segment of mice sperm flagella. (F) Electron micrograph of mitochondria at the middle segment of mice sperm. (G) Electron micrograph of mice sperm head; CCDC157 mutant mice sperm heads show abnormalities such as cytoplasmic residue, isolated acrosome nuclei, and irregular nucleus. −/− represents CCDC157 knockout homozygous mice, and +/+ represents wild-type mice.

FIG. 5 shows the comparison of spermatogenesis stages of seminiferous tubules in CCDC157 knockout homozygous mice and wild-type mice. (A) TEM image; the morphology of the mutant round sperm 5-8 was not significantly different from that of the wild-type mice; but the elongated sperm 13 was significantly different from that of the wild-type mice, with round and abnormal nuclei. (B) HE staining image; seminiferous tube stages VIII-XII, it can be seen in stage VIII that many elongated sperm in CCDC157 mutants cannot be released into the lumens of seminiferous tubules (arrows), and there were many abnormal sperm cell nuclei in mutant XI-XII (short arrows). −/− represents CCDC157 knockout homozygous mice, and +/+ represents wild-type mice.

FIG. 6 shows transcriptome analysis on CCDC157 in patients with non-obstructive azoospermia (NOA). 2 cases were obstructive azoospermia, with normal spermatogenesis (NS), 2 cases were mature arrest (MA), and only sertoli cells (SCOS, sertoli-cells only syndrome) were found in the testicular biopsy tissues of the other 2 cases. The expression of CCDC157 in patients with non-obstructive azoospermia (NOA) was down-regulated.

### DESCRIPTION OF EMBODIMENTS

The present application provides use of a new mutation site of CCDC157 (MIF515 mutation), that is, a mutant gene/protein as a molecular marker in the diagnosis of male infertility diseases. The MIF515 mutation occurs in a nucleotide sequence shown in SEQ ID NO. 1 or the MIF515 mutation occurs in an amino acid sequence of a polypeptide shown in SEQ ID NO. 2. Disclosed is use of the CCDC157-MIF515 mutation site as a molecular marker in the preparation of a kit for diagnosing male infertility, which provides a new molecular marker and therapeutic target for the existing kit for diagnosing male infertility.

The present application further provides use of the CCDC157 gene/protein. The CCDC157 gene/protein can be used as a molecular marker in the preparation of a kit for diagnosing male infertility, and can also be used as a drug target to prepare a product having one or more functions of preventing male infertility, treating male infertility, treating sperm dysfunction, promoting sperm maturation, improving sperm motility, increasing sperm count, repairing abnormal sperm morphology, repairing a sperm head structure, and improving male fertility.

The present application will be further described in detail through specific embodiments with reference to the accompanying drawings. The described embodiments are only some of the embodiments of the present application, rather than all the embodiments. Based on the embodiments of the present application, all other embodiments obtained by those of ordinary skill in the art without any creative efforts shall fall within the protection scope of the present application.

The experimental methods in the following embodiments are conventional methods, unless otherwise specified; and the experimental materials used in the following examples are purchased from conventional biochemical reagent stores, unless otherwise specified.

The experimental animals used in the following examples are all raised and bred in the Animal Experiment Center of Zhejiang University, and water and food are freely available during experiments.

### Example 1 Screening of CCDC157-MIF515 Mutant Gene Patients in Asthenospermia Patients and their Semen Analysis

1) This study was approved by the ethics committee of the Obstetrics and Gynecology Hospital Affiliated to Zhejiang University School of Medicine, and the patients were informed and consented. 50 cases of semen were collected from patients with asthenospermia in the Obstetrics and Gynecology Hospital Affiliated to Zhejiang University School of Medicine, and patients with organic diseases of the reproductive system, patients with untreated endocrine disorders, patients abusing drugs or alcohol within two years, and patients with abnormal semen caused by known diseases such as abnormal chromosomal and AZF, cryptorchidism, or mumps were excluded.

The patients with asthenospermia masturbated to obtain semen, and DNA of the patient' semen was extracted with a TIANamp Micro DNA Kit (genomic DNA was extracted from micro-tissues according to the sixth item of the instruction).

3 pairs of primers were designed for two conserved functional domains of CCDC157-SMC domain (i.e. MIF515 site), as follows:

h CCDC157-F-1:
(SEQ ID NO. 3)
5′-GAACCTGCCCTCGTCCTTAG-3′
h CCDC157-R-1:
(SEQ ID NO. 4)
5′-ATCACAGTGTCACCACCGGA-3′
h CCDC157-F-2:
(SEQ ID NO. 5)
5′-AGGTGTAGACACATGGCAGC-3′
h CCDC157-R-2:
(SEQ ID NO. 6)
5′-AAAGGTGAGTCAACCCCCAC-3′
h CCDC157-F-3:
(SEQ ID NO. 7)
5′-TACCCTTCCACGCATGTGAC-3′
h CCDC157-R-3:
(SEQ ID NO. 8)
5′-GGCTTTAACACCCTTGCTGC-3′

Taking the patient's genomic DNA as a template, an SMC domain of a CCDC157 gene (i.e. MIF515 site) was amplified by polymerase chain reaction (PCR) and sent for sequencing. After the sequencing results were returned, sequence comparisons were performed to screen patients with CCDC157-MIF515 mutant genes.

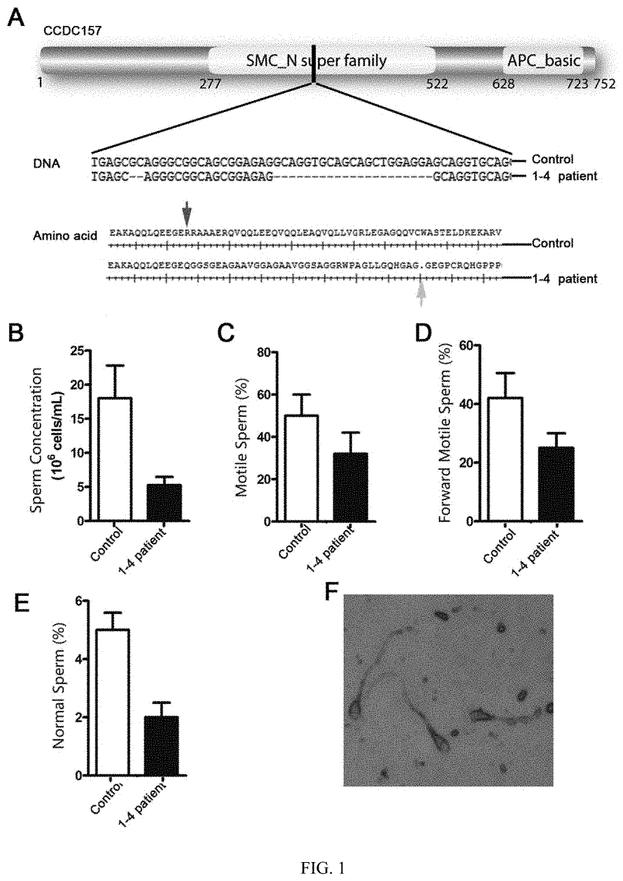

The results were as follows: 1 case of patient (1-4 patients) with CCDC157-MIF515 mutation defective gene was screened out, and the CCDC157-MIF515 mutation showed base deletions at the middle part shown in SEQ ID NO.1, that is, GC deletions on positions 15488-15489 and GCAGGTGCAGCAGCTGGAGGA (SEQ ID NO. 9) deletions on positions 15507-15527 of a CCDC157 gene sequence; or its amino acid sequence showed that R was mutated to Q in the amino acid on position 387 of a CCDC157 polypeptide, and the amino acids on positions 388-424 were mutated from RAAAERQVQQLEEQVQQLEAQVQLLVGRLEGAGQQVC (SEQ ID NO.10) to GGSGEAGAAVGGAGAAVGGSAGGRWPAGLLGQHGAG (SEQ ID NO. 11), and finally the amino acid W on position 425 was mutated to a terminator, which terminated the gene early (the A of FIG. 1).

2) The patient's sperm status was analyzed; his semen was collected after 3-5 days of abstinence, liquefied at 37° C. for 30 minutes, and then tested with CASA for semen volume, concentration, sperm morphology, vitality and viability.

The results were as follows: the sperm concentration, the percentage of motile sperm, the percentage of forward motile sperm, and the percentage of normal morphological sperm of the patient with the CCDC157-MIF515 mutant gene decreased (the patient was tested twice for taking average values) (the B-E of FIG. 1).

### Example 2 Obtaining of CCDC157 Gene Knockout Mice

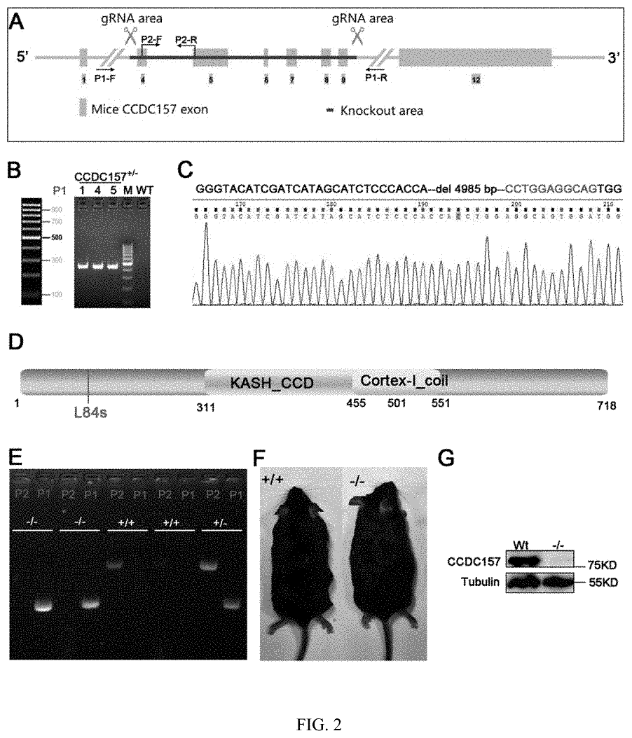

1) Two sgRNA sequences for a CCDC157 gene (No. 216516 in the NCBI database) were designed using CRISPR/Cas9 technology through the CRISPR online website (http://crispr.mit.edu). The design strategy was shown in the A of FIG. 2, gRNA1 (SEQ ID NO. 12): CTCTGAGAGCGGCCTATGGTGGG; and gRNA2 (SEQ ID NO. 13): GGGAGGATCCATCCAACCTAGGG (the two gRNAs were antisense strands of matched genes). After synthesis and annealing, the CCDC157 gene was connected to a pX458 vector expressing a Cas9 protein.

2) The pX458 vector was transferred to embryonic stem cells, which were then transferred to a culture dish after flow screening and cultured for 24 hours. Monoclonal selection and genotype identification were performed after the culture was completed.

3) C57BL/6N female mice superovulated by hormone treatment were mated with wild-type male mice in advance to obtain a large number of blastocyst cells (ligated male mice were also mated with C57BL/6N female mice to produce pseudo-pregnant female mice). The genetically modified embryonic stem cells selected in the previous step were injected into the blastocysts obtained in this step, and after a short period of in vitro culture, the blastocysts were transferred back to the uteri of the pseudo-pregnant female mice to obtain F0 generation gene mutant mice.

4) Genotypes of the F0 generation mice were identified by a PCR method, the identification system was 25 μl, specifically as follows:

DEPC water 14.5 μl
DNA 0.5 μl
10 × Buffer 2.5 μl
2.5 mM dNTP Mix 4.0 μl
Primer-F/R 1.5 μl/1.5 μl
rTaq 0.5 μl

The reaction conditions were: pre-denaturation at 95° C. for 3 min, cycle number 1; denaturation at 95° C. for 30 seconds, annealing for 30 s, extension at 72° C. for 1 min/1000 bp, cycle number 33; extension at 72° C. for 10 min, cycle number 1; 12° C. The primer sequences were as follows:

CCDC157P-F1:
(SEQ ID NO. 14)
5′-GCTCTGCCTCCTTCTGAGTTAG-3′
CCDC157P-R1:
(SEQ ID NO. 15)
5′-GTTTGTCTTCTGACCACACTCC-3′
CCDC157P-F2:
(SEQ ID NO. 16)
5′-CTCGTCTCAATAAACATGTGGG-3′
CCDC157P-R2:
(SEQ ID NO. 17)
5′-CCACCTTTGTCTTTAGGTCACA-3′

In the wild-type mice, when CCDC157P-F1/R1 (P1) were used as primers, any band cannot be amplified; when CCDC157P-F2/R2 (P2) were used as primers, 904 bp bands can be amplified; after the CCDC157 gene was knocked out from the mice, 444 bp bands were amplified with the primers P1, and any band cannot be amplified with the primers P2. Thus, F1 mice with the CCDC157 knockout allele can be identified. The results of sequencing analysis on the products amplified with the primers P1 showed that the knockout of the CCDC157 gene resulted in a 4985 bp deletion and an 11 bp insertion (the C of FIG. 2). The CCDC157 protein framework was shown in the D of FIG. 2; and the CCDC157 mutant allele terminated after the 84th leucine.

5) The CCDC157 knockout heterozygous F1 mice mated with each other to obtain CCDC157 knockout homozygous F2 mice (hereinafter referred to as CCDC157^{-/-} mice) and wild-type mice (hereinafter referred to as CCDC157^{+/+} mice).

6) Western blot analysis was performed on the CCDC157 protein expression in the tested mice. The details were as follows: the mice to be tested were dissected to collect testicular tissues, the testicular tissues were ground on ice with a cell lysis solution (50 mM Tris.HCl pH8.0, 150 mM NaCl, 1% IGEPAL CA-630, 0.5% Sodium Deoxycholate, 0.1% SDS, protease inhibitor), the ground testicular tissues were placed on the ice for 30 min and then centrifuged at 4° C. and 13000 rpm for 30 min, the supernatant was pipetted, and the protein concentration was measured by a BCA method. 40 μg of protein was added to 2×Lading Buffer, denatured at 95° C. for 5 min, centrifuged and then loaded to 10% SDS-PAGE gel. The sample was transferred to a PVDF membrane (ImmobilonP, Millipore, Billerica, Mass.). The PVDF membrane was sealed for 1 h with 5% skimmed milk powder (TBST contained 0.01% Tween20); a primary antibody [rabbit polyclonal CCDC157 antibody (dilution ratio 1:100, GeneTeX company, article number: GTX45090) or a monoclonal mice β-tubulin antibody (dilution ratio 1: 2000)] was added, followed by overnight incubation at 4° C.; the membrane was washed 3 times with TBST, 10 min each time; a secondary antibody was added, followed by incubation at room temperature for 1 h, and TBST membrane washing 3 times, 10 min each time. Development and exposure were carried out using an ECL kit (37071, Pierce, Thermo Fisher Scientific).

The results were as follows: CCDC157^{+/+} mice had CCDC157 protein bands; and CCDC157^{-/-} mice did not have CCDC157 protein bands (the G of FIG. 2), indicating that the CCDC157 gene was knocked out.

### Example 3 CCDC157 Gene Knockout Caused Infertility in Male Mice

1) The growth and development of CCDC157−/− mice and CCDC157+/+ mice were observed and counted. The results showed that there was no difference in survival rate, appearance (as shown in the F of FIG. 2) and overall behavior between CCDC157^{-/-} mice and CCDC157^{+/+} mice; the male CCDC157^{-/-} mice were completely sterile, while the fertility of the female CCDC157^{-/}mice was not affected.

The above results indicated that the CCDC157 gene knockout caused male sterility in mice.

2) 3 male CCDC157^{-/-} mice were mated with several female wild-type mice for 3 months. Whether sperm plugs were present in the reproductive tract of the female wild-type mice was observed during the mating. The results were as follows: sperm plugs were present in the reproductive tract of the female wild-type mice, indicating that the mating was normal; but no offspring was born.

The above results indicated that the CCDC157 gene knockout affected the fertilization process.

3) The gonads of the CCDC157^{-/-} mice were observed. The details were as follows: wild-type and CCDC157 knockout mice grown to 8-12 weeks were selected and sacrificed at their necks; the mice's abdominal hair was wiped with an alcohol cotton ball, the mice were dissected from the abdomen with dissecting scissors, the gonads of the mice were clipped with tweezers, the testis, epididymis, and seminal vesicles were separated, and the weight was measured.

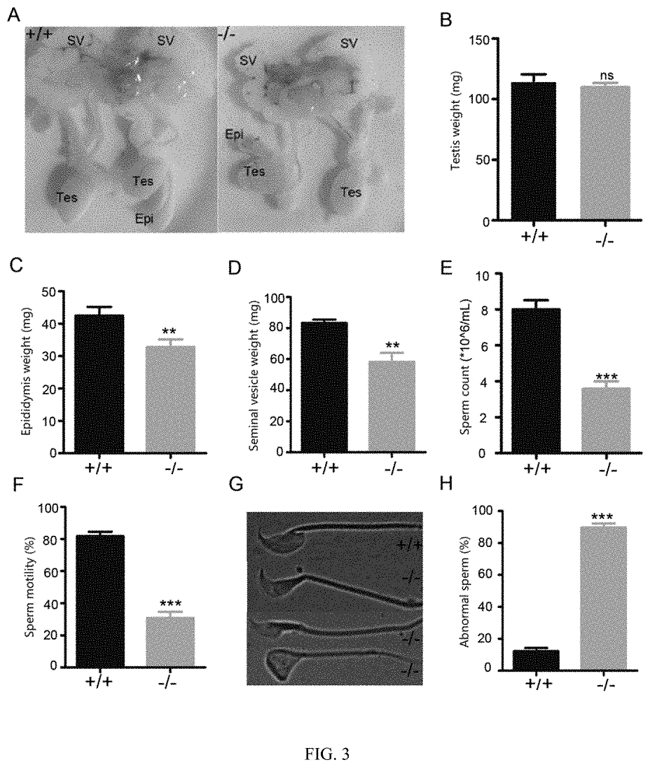

The results were as follows: as shown in the A-D of FIG. 3, there was no significant difference in structures of the gonads of the male CCDC157^{-/-} mice and CCDC157^{+/+} mice. The size and weight of the testes of the CCDC157−/− mice were not significantly different from those of the wild-type mice; however, the epididymis and seminal vesicles of the CCDC157−/− mice were significantly smaller than those of the wild-type mice.

4) The vitality and quantity of sperm were tested by CASA experiments. The details were as follows: mice epididymis was placed in a CASA buffer, the epididymis was cut and incubated in a 37° C., 5% CO₂ incubator for 5 minutes, 60 minutes, 90 minutes and 120 minutes respectively to collect sperm, and the vitality of the sperm was analyzed.

Sperm count: mice epididymis was placed in a CASA buffer, the epididymis was cut (the number of cuts was recorded and the number and size of cuts should be uniform) and incubated at 37° C. for 30 minutes, and after the sperm swam out, the sperm was filtered with a 40 μm filter screen. The sperm was diluted 10 times and then mixed well. 10 μL was dropped onto a Biorad cell counting plate. Images were shot with a microscope, and the number of cells in each field of view was analyzed by Image J. The corresponding sperm count was calculated according to the thickness of the counting plate, the conversion formula for the shooting field of view, and the dilution factor and total volume of the sperm.

Formula of the CASA buffer: 120 mM NaCl, 4.8 mM KCl, 1.2 mM MgSO₂, 1 mM CaCl₂, 1.2 mM KH₂PO₄, 21 mM sodium DL-lactate (Na-dl-lactate), 5 mM glucose, 25 mM NaHCO₃, 0.25 mM sodium pyruvate (Na-pyruvate), 0.4 m/mL phenol red, 3 mg/mL bovine serum albumin (BSA V).

The results were as follows: compared with the CCDC157^{+/+} mice, the quantity of sperm and the percentage of viable sperm in the tail of epididymis of the CCDC157^{-/-} mice were significantly reduced (the E and F of FIG. 3).

5) The sperm morphology was observed under an optical microscope. The details were as follows: the epididymis was torn off, cut several times with scissors, flicked, and placed at room temperature or 37° C. for 5 minutes until the sperm swam out, and the supernatant was pipetted with a disposable pipette and filtered with a 100 μm cell strainer. 5 μL of the above suspension was dropped to one end of a glass slide coated with D-polylysine. A push slide was placed at a 30-degree angle with the glass slide, placed directly in front of the semen, and moved slightly back to contact semen droplets. It can be seen that the droplets were spread along the lower edge of the push slide to drive the scattered semen line. The push slide was pulled at a constant speed without contacting the glass slide to form a semen smear. The morphology and abnormality percentage of the sperm was observed under the optical microscope.

The results were as follows: the heads of the CCDC157^{-/-} mice sperm were mostly deformed, round and irregular (the G of FIG. 3); but the tails of the sperm did not appear to be abnormal, and their lengths were also normal (the G of FIG. 3); compared with the CCDC157^{+/+} mice, the percentage of sperm deformities at the tail of epididymis of the CCDC157^{-/-} mice was significantly increased (the H of FIG. 3).

### Example 4 CCDC157 Gene Knockout Caused Abnormal Sperm Morphology

1) The epididymis of adult mice to be tested was dissected. The mice to be tested were male CCDC157^{+/+} mice and male CCDC157^{-/-} mice aged 8-12 weeks. Paraffin sections were prepared (fixation-dehydration-transparent tissue-waxing-embedding-slicing-extension-pasting), and the epididymis of the mice to be tested was analyzed by HE staining (dewaxing-rehydration-hematoxylin staining-washing-color separation with hydrochloric acid and alcohol-washing-Eosin staining-dehydration-transparency-sealing).

The results were as follows: the amount of sperm at the tail of epididymis of the CCDC157 knockout mice was significantly smaller than that of the wild-type mice, and many sperm with head deformities can be seen at magnification.

2) The structure of sperm in the tail of epididymis of the mice was analyzed by transmission electron microscopy.

The details were as follows: the sample was fixed overnight at 4° C. in a 2.5% glutaraldehyde solution (diluted with 0.1M pH 7.2 PBS). The fixative solution was pipetted, and the sample was rinsed three times with 0.1M pH 7.2 PBS for 15 min each; the sample was fixed with a 1% osmium acid solution for 1-2 h; the fixative solution was removed, and the sample was rinsed three times with 0.1M pH 7.2 PBS for 15 min each; gradient dehydration was carried out with ethanol: the sample was dehydrated with an ethanol solution having gradient concentrations (including 30%, 50%, 70%, 80%, 90% and 95%) for 15 min in each concentration, and then treated with 100% ethanol for 20 min; and finally the sample was treated with pure acetone for 20 min. Gradient penetration with an embedding agent: \textcircled{1} the sample was treated with a mixture of embedding agent and acetone (V/V=1/1) for 1 h; \textcircled{2} the sample was treated with a mixture of embedding agent and acetone (V/V=3/1) for 3 h; and \textcircled{3} the pure embedding agent penetrated the sample overnight: in this step, the sample should be transferred to a new dry tube, and the pure embedding agent was put in the new tube. The penetrated sample was dispensed into 0.5 mL eppendorf tubes and embedded, and heated at 70° C. overnight. The sample was oriented. The sample was cut with an ultra-microtome and placed on a copper mesh. The sample was stained with uranyl acetate and observed under an electron microscope.

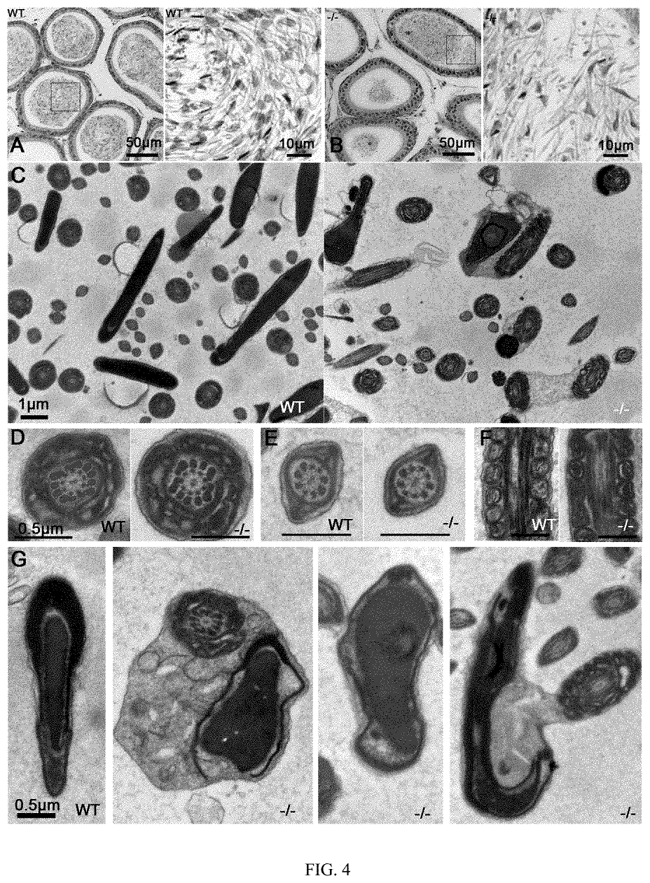

The results were as follows: the quantity of sperm of the CCDC157^{-/-} mice was less than that of the CCDC157^{+/+} mice (the A and B of FIG. 4). The middle segment, main segment and mitochondrion structure of the sperm tail of the CCDC157^{-/-} mice were not affected (the D-F of FIG. 4); but the head of the sperm was obviously deformed, showing phenotypes such as irregular nuclei, abnormal acrosome structure, isolated acrosome nuclei, and cytoplasmic residue (the G of FIG. 4).

### Example 5 CCDC157 Gene Knockout Affected Sperm Differentiation, but Did not Affect Early Sperm Development

1) The testes of adult mice to be tested were dissected. The mice to be tested were male CCDC157^{+/+} mice and male CCDC157^{-/-} mice aged 8-12 weeks. The testicular tissues of the mice were analyzed under an electron microscope. The specific operation was the same as that in Example 2.

2) The testes of adult mice to be tested were dissected. The mice to be tested were male CCDC157^{+/+} mice and male CCDC157^{-/-} mice aged 8-12 weeks. The testicular tissues of the mice were analyzed by HE staining. The specific operation was the same as that in Example 2.

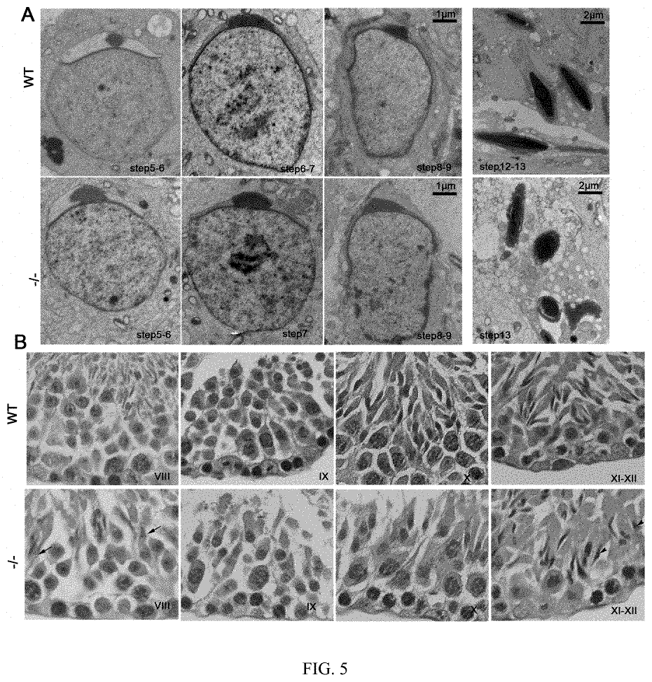

The results were as follows: the round sperm of the CCDC157^{-/-} mice after meiosis was normal in steps 1-9 (the A of FIG. 5), but sperm cells were abnormal in step 13, so the abnormality appeared in steps 9-12 of development of the sperm cells. The HE staining analysis on the seminiferous tubules of the testis showed that the release of some elongated sperm in the CCDC157 mutants was also abnormal. In stage 8 of the seminiferous tubules, many elongated sperm cannot be released into the lumens of the seminiferous tubules (arrows in the B of FIG. 5). The above results indicated that the deletion of CCDC157 in mice did not affect the early stage of sperm development, but affected the differentiation of sperm.

The above results showed that the CCDC157-MIF515 mutation site can be used as a molecular marker for the diagnosis of male infertility.

### Example 6 Expression of CCDC157 in Patients with Non-Obstructive Azoospermia (NOA) was Down-Regulated

This study was approved by the ethics committee of the Obstetrics and Gynecology Hospital Affiliated to Zhejiang University School of Medicine, and the patients were informed and consented. Human testicular tissues were collected during testicular biopsy in the Obstetrics and Gynecology Hospital Affiliated to Zhejiang University School of Medicine. The testicular tissues of 6 patients with azoospermia were collected. Among them, 2 cases were obstructive azoospermia (OA), with normal spermatogenesis (NS); 2 cases were mature arrest (MA); and only sertoli cells (SCOS, sertoli-cells only syndrome) were found in the testicular biopsy tissues of the other 2 cases. RNA was extracted from the testicular tissues, specifically as follows: the testicular tissues were washed with PBS and then placed in RNase free EP tubes, an appropriate amount of Triozol was added to each sample (1 ml of Trizol was added to 50-100 mg of sample) (Sigma), and the sample was broken and mixed at room temperature. The sample was centrifuged at 4° C. and 12000 rpm for 10 min, the supernatant was transferred to a new RNase free EP tube, and the undissolved tissues were discarded. The sample was stood at room temperature for 5 min to ensure that the nucleoprotein complex was fully dissociated. An appropriate amount of chloroform was added according to the amount of Trizol initially added (0.2 ml of chloroform was added to 1 ml of Trizol), followed by vigorous shaking for 15 seconds and standing at room temperature for 2 to 3 min. The sample was centrifuged at 4° C. and 12000 rpm for 15 min, and the sample was delaminated. The upper aqueous phase was carefully pipetted into a new EP tube (approximately 80% of the upper aqueous phase was pipetted), without pipetting the intermediate phase and the lower red organic phase. An appropriate amount of 100% isopropanol was added according to the amount of Trizol initially added (0.5 ml of isopropanol was added to 1 ml of Trizol), followed by standing at room temperature for 10 min. The sample was centrifuged at 4° C. and 12000 rpm for 10 min. The supernatant was discarded and the white RNA precipitate at the bottom of the tube was retained. 75% ethanol (prepared with DEPC-H₂O, 1 ml of 75% ethanol was added to per 1 ml of Trizol) was added, followed by shaking upside down several times, centrifugation at 4° C. and 7500 rpm for 5 min, and discarding of the supernatant. This step can be repeated once. The lid was opened and standing was carried out at room temperature to dry the RNA precipitate. An appropriate amount of RNase-free water was added, followed by pipetting several times, to re-dissolve RNA.

The RNA samples were sent to the Beijing Genomics Institute for transcriptome sequencing.

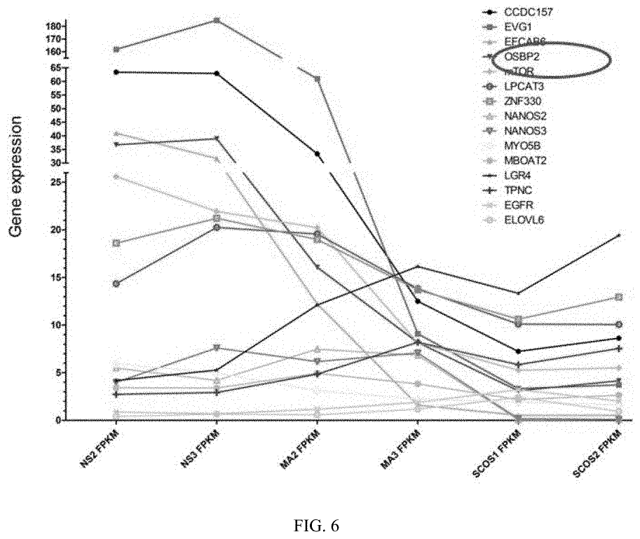

The results were as follows: as shown in FIG. 6, the expression level of CCDC157 was significantly reduced in NOA patients and SCOS patients. In addition, the expression levels of some lipid metabolism-related factors, such as OSBP2, were also significantly reduced in NOA patients and SCOS patients. This showed that the expression of the CCDC157 gene/protein had an important impact on male infertility, and the increase in the activity and/or expression of the CCDC157 protein can prevent and/or treat male infertility.

Sequence Listing
1
736
DNA
Homo sapiens
1
agagcagagg aaagacctga cgcgcctcag taagcatatg gaggccctca gggcccagct 60
ggaggaggct gaagggcaga aggatggcct gaggaagcag gcgggcaagc tggagcaggc 120
gctgaaacag gagcagagag caccacgaca acaggccgag gaggatgacc agtgcctatc 180
tgagtcggag cacgacaaac agcagctgct cacagaaaca agtgacctaa agacaaagat 240
ggccaccctg gagagagaac tgaaacagca gcgggagtcc acacaggctg tggaggcaaa 300
ggcccagcag ctgcaggagg aaggtgagcg cagggcggca gaggagaggc aggtgcagca 360
gctggaggag caggtgcagc agttggaggc gcaggtgcag ctgttggtgg gtcggctgga 420
gggcgctggc cagcaggtct gctgggccag cacggagctg gataaggaga aggcccgtgt 480
cgacagcatg gtccgccacc aggagtctct gcaggccaag cagcgagccc tgctaaagca 540
gctggacagc ctggaccagg aacgtgagga gctgcggggc agcctggacg aggctgaggc 600
ccagcgggcc cgcgtggagg agcagctgca gagcgagcgg gagcaggggc aatgccagct 660
cagggcccag caggagctgc tgcagagcct gcagagggag aagcaaggcc tggagcaggc 720
gactacggac ctgcgg 736
agagcagaggaaagacctgacgcgcctcagtaag
catgtggaggccctcagggcccagctggaggag
gctgaagggcagaaggatggcctgaggaagcag
gcgggcaagctggagcaggcgctgaaacaggag
cagggggcacggcggcgacaggcggaggaggat
gagcagtgcctgtctgagtgggagcacgacaaa
cagcagctgctcacagaaacaagtgacctaaag
acaaagatggccaccctggagagagaactgaaa
cagcagcgggagtccacacaggctgtggaggca
aaggcccagcagctgcaggaggaaggtgagcgc
agggcggcagcggagaggcaggtgcagcagctg
gaggagcaggtgcagcagttggaggcgcaggtg
cagctgttggtgggtcggctggagggcgctggc
cagcaggtctgctgggccagcacggagctggat
aaggagaaggcccgtgtcgacagcatggtccgc
caccaggagtctctgcaggccaagcagcgagcc
ctgctaaagcagctggacagcctggaccaggaa
cgtgaggagctgcggggcagcctggacgaggct
gaggcccagcgggcccgcgtggaggagcagctg
cagagcgagcgggagcaggggcaatgccagctc
agggcccagcaggagctgctgcagagcctgcag
agggagaagcaaggcctggagcaggcgactacg
gacctgcgg
2
276
PRT
Homo sapiens
2
Ala Glu Gln Arg Lys Asp Leu Thr Arg Leu Ser Lys His Val Glu 15
Ala Leu Arg Ala Gln Leu Glu Glu Ala Glu Gly GIn Lys Asp Gly 35
Leu Arg Lys Gln Ala Gly Lys Leu Glu Gln Ala Leu Lys Gln Glu 45
Gln Gly Ala Arg Arg Arg Gln Ala Glu Glu Asp Glu Gln Cys Leu 60
Ser Glu Tre Glu His Asp Lys Gln Gln Leu Leu Thr Glu Thr Ser 75
Asp Leu Lys Thr Lys Met Ala Thr Leu Glu Arg Glu Leu Lys Gln 90
Gln Arg Glu Ser Thr Gln Ala Val Glu Ala Lys Ala Gln Gln Leu 105
Gln Glu Glu Gly Glu Arg Arg Ala Ala Ala Glu Arg Gln Val Gln 120
Gln Leu Glu Glu Gln Val Gln Gln Leu Glu Ala Gln Val Gln Leu 135
Leu Val Gly Arg Leu Glu Gly Ala Gly Gln Gln Val Cys Try Ala 155
Ser Thr Glu Leu Asp Lys Glu Lys Ala Arg Val Asp Ser Met Val 165
Arg His Gln Glu Ser Leu Gln Ala Lys Gln Arg Ala Leu Leu Lys 150
Gln Leu Asp Ser Leu Asp Gln Glu Arg Glu Glu Leu Arg Gly Ser 155
Leu Asp Glu Ala Glu Ala Gln Arg Ala Arg Val Glu Glu Gln Leu 210
Gln Ser Glu Arg Glu Gln Gly Gln Cys Gln Leu Arg Ala Gln Gln 225
Glu Leu Leu Gln Ser Leu Gln Arg Glu Lys Gln Gly Leu Glu Gln 240
Ala Thr Thr Asp Leu Arg Leu Thr Ile Leu Glu Leu Glu Arg Glu 255
Leu Glu Glu Leu Lys Glu Arg Glu Arg Leu Leu Val Ala Phe Pro 270
Asp Leu His Arg Pro Thr 275
AlaGluGlnArgLysAspLeuThrArgLeuSer
LysHisValGluAlaLeuArgAlaGlnLeuGlu
GluAlaGluGlyGlnLysAspGlyLeuArgLys
GlnAlaGlyLysLeuGluGlnAlaLeuLysGln
GluGlnGlyAlaArgArgArgGlnAlaGluGlu
AspGluGlnCysLeuSerGluTrpGluHisAsp
LysGlnGlnLeuLeuThrGluThrSerAspLeu
LysThrLysMetAlaThrLeuGluArgGluLeu
LysGlnGlnArgGluSerThrGlnAlaValGlu
AlaLysAlaGlnGlnLeuGlnGluGluGlyGlu
ArgArgAlaAlaAlaGluArgGlnValGlnGln
LeuGluGluGlnValGlnGlnLeuGluAlaGln
ValGlnLeuLeuValGlyArgLeuGluGlyAla
GlyGlnGlnValCysTrpAlaSerThrGluLeu
AspLysGluLysAlaArgValAspSerMetVal
ArgHisGlnGluSerLeuGlnAlaLysGlnArg
AlaLeuLeuLysGlnLeuAspSerLeuAspGln
GluArgGluGluLeuArgGlySerLeuAspGlu
AlaGluAlaGlnArgAlaArgValGluGluGln
LeuGlnSerGluArgGluGlnGlyGlnCysGln
LeuArgAlaGlnGlnGluLeuLeuGlnSerLeu
GlnArgGluLysGlnGlyLeuGluGlnAlaThr
ThrAspLeuArg
AEQRKDLTRLSKHVEALRAQLEEAEGQKDGLR
KQAGKLEQALKQEQGARRRQAEEDEQCLSEWE
HDKQQLLTETSDLKTKMATLERELKQQRESTQ
AVEAKAQQLQEEGERRAAAERQVQQLEEQVQQ
LEAQVQLLVGRLEGAGQQVCWASTELDKEKAR
VDSMVRHQESLQAKQRALLKQLDSLDQEREEL
RGSLDEAEAQRARVEEQLQSEREQGQCQLRAQ
QELLQSLQREKQGLEQATTDLRLTILELEREL
EELKERERLLVAFPDLHRPT

What is claimed is:

1. A CCDC157-MIF515 mutant gene, wherein the CCDC157-MIF515 mutant gene is produced by heterozygous mutation of a CCDC157 gene on a third exon, and a nucleotide sequence of the mutant gene shows GC deletions on positions 15488-15489 and base deletions on positions 15507-15527 of a CCDC157 gene sequence; or an amino acid sequence of the mutant gene shows that R is mutated to Q in the amino acid on position 387 of a CCDC157 polypeptide, and the amino acids on positions 388-424 are mutated to an amino acid sequence shown in SEQ ID NO.11, and the amino acid Won position 425 is mutated to a terminator.

2. Use of the CCDC157-MIF515 mutant gene according to claim 1 as a molecular marker in the preparation of a kit for diagnosing male infertility.

3. The use according to claim 2, wherein the test kit comprises primers for testing a CCDC157 protein or a CCDC157 DNA or RNA sequence.

4. A kit for diagnosing male infertility, wherein the kit comprises a reagent for detecting a nucleotide sequence shown in SEQ ID NO. 1 or an amino acid sequence shown in SEQ ID NO. 2, and a carrier recording a judgment criterion; the judgment criterion is: if the CCDC157-MIF515 mutant gene/protein is present in the sperm of a test subject, the test subject is or is suspected to be a male infertile patient; wherein, the CCDC157-MIF515 mutant gene is produced by heterozygous mutation of a CCDC157 gene on a third exon, and a nucleotide sequence of the mutant gene shows GC deletions on positions 15488-15489 and base deletions on positions 15507-15527 of a CCDC157 gene sequence; or an amino acid sequence of the mutant gene shows that R is mutated to Q in the amino acid on position 387 of a CCDC157 polypeptide, and the amino acids on positions 388-424 are mutated to an amino acid sequence shown in SEQ ID NO.11, and the amino acid W on position 425 is mutated to a terminator.

5. The kit according to claim 4, wherein the reagent comprises 3 pairs of primers, and the nucleotide sequences of the primers are shown in SEQ ID NO. 3 to SEQ ID NO. 8.
